(19) United States
(12) Patent Application Publication
(10) Pub. No.: US 2018/0132709 A1
(43) Pub. Date: May 17, 2018

(54) Laryngoscope

(71) Applicant: Tien-Sheng Chen, Taipei City (TW)

(72) Inventor: Tien-Sheng Chen, Taipei City (TW)

(21) Appl. No.: 15/702,886

(22) Filed: Sep. 13, 2017

(30) Foreign Application Priority Data
Nov. 16, 2016 (TW) 105137437

(51) Int. Cl. A61B 1/267; A61B 1/04; A61B 1/00

(52) U.S. Cl. CPC A61B 1/267; A61B 1/00154; A61B 1/00045; A61B 1/04

(57) ABSTRACT

A laryngoscope with a blade, a camera, a display and a guiding stylet is disclosed. The blade has a camera accommodation portion, an endotracheal tube passage and a guide channel. The camera is situated in the camera accommodation portion and electrically connected with the display. The guiding stylet is situated inside an endotracheal tube and has a guiding unit which is detachably situated within the guide channel and moves relative to the guide channel.

### BACKGROUND OF THE INVENTION

### 1. Field of the Invention

The present invention relates to a laryngoscope; more particularly, the present invention relates a laryngoscope having a guide channel and a guiding stylet.

### 2. Description of the Related Art

For patients with respiratory termination, tracheal intubation is a necessary means for maintaining respiratory function. In order to maintain the patient's life, medical personnel must insert an endotracheal tube into the patient's trachea to provide oxygen in a short time. However, because the upper respiratory tract structures of patients vary due to different ages or body types, sometimes the medical personnel cannot easily or directly observe a patient's tracheal position. Therefore, on most of the laryngoscopes currently in use, a camera device is installed on a side of an endotracheal tube channel. After the medical personnel observe the patient's endotracheal opening through the camera device, the endotracheal tube is inserted through the endotracheal tube passage disposed at the side of the camera device and into the patient's trachea.

However, there are still inconveniences to be overcome in using the laryngoscope of the prior art. Sometimes, because of differences in the body structures of patients, the endotracheal tube passage is obstructed by hypertrophic soft tissue or swollen tonsils of the patient; thus, the medical personnel cannot successfully insert the endotracheal tube through the endotracheal tube passage into the trachea, or the soft tissue in the throat may be punctured by the tip of the endotracheal tube when the tube is inserted. As the current laryngoscope does not allow control of the direction of the endotracheal tube, the endotracheal tube will be inserted arbitrarily into the patient's upper respiratory tract during endotracheal tube intubation. Such an insertion may cause injury to the patient's upper respiratory tract during the endotracheal tube intubation. In the medical literature, the problems of the endotracheal tube puncturing the patient's respiratory tract is addressed, so the tracheal insertion tool of the prior art must be improved.

### SUMMARY OF THE INVENTION

It is an object of the present invention to provide a laryngoscope with a guide channel and a guiding stylet.

To achieve the abovementioned object, the laryngoscope of the present invention comprises a blade, a camera, a display and a guiding stylet. The blade comprises a camera accommodation portion, an endotracheal tube passage and a guide channel, wherein the guide channel is situated between the camera accommodation portion and the endotracheal tube passage. The camera is situated at the camera accommodation portion. The display is electrically connected to the camera. The guiding stylet is situated inside the endotracheal tube and comprises a guiding unit which is detachably situated inside the guide channel and movable relative to the guide channel.

With the design of the guide channel and the guiding stylet, it is possible to limit the movement range of the guiding stylet tip and control the direction of the front end of the endotracheal tube. Thus, the situation of the tip of the guiding stylet or the front end of the endotracheal tube puncturing the patient's tissue when the endotracheal tube is inserted into the patient's trachea as mentioned in the prior art can be avoided, and the accuracy of the insertion of the endotracheal tube into the trachea can also be improved.

Other objects, advantages and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIG. 1 illustrates a schematic diagram of one embodiment of the laryngoscope of the present invention.

FIG. 2A illustrates an explosion schematic of the blade, the camera and the guiding stylet.

FIG. 2B illustrates a partial magnification diagram of the guiding stylet.

FIG. 3 is a schematic diagram of the guiding stylet moving in the guide channel.

FIG. 4 is a schematic diagram of the guiding stylet moving in the guide channel.

FIG. 5 is a schematic diagram of the guiding stylet separating from the guide channel.

### DETAILED DESCRIPTION OF THE PREFERRED EMBODIMENT

These and other objects and advantages of the present invention will become apparent from the following description of the accompanying drawings, which disclose several embodiments of the present invention. It is to be understood that the drawings are to be used for purposes of illustration only and not as a definition of the invention.

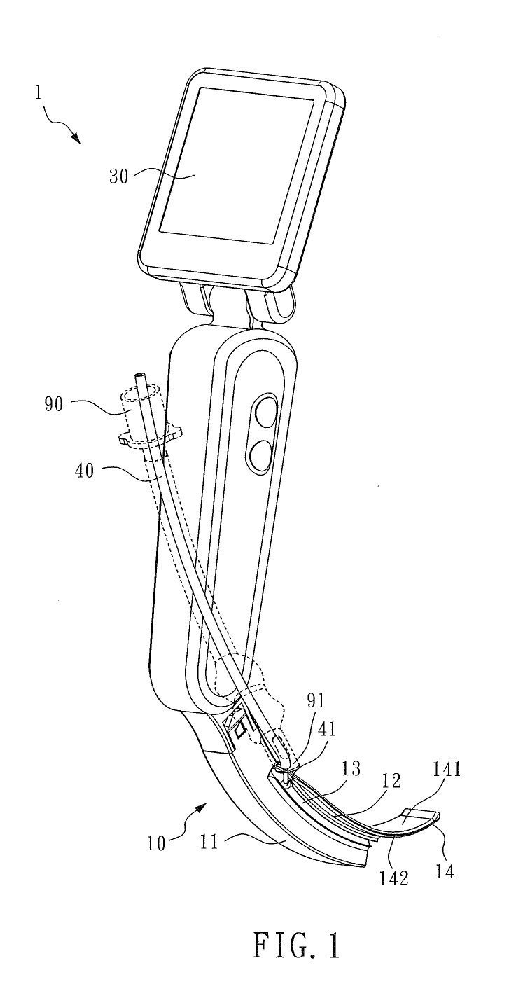

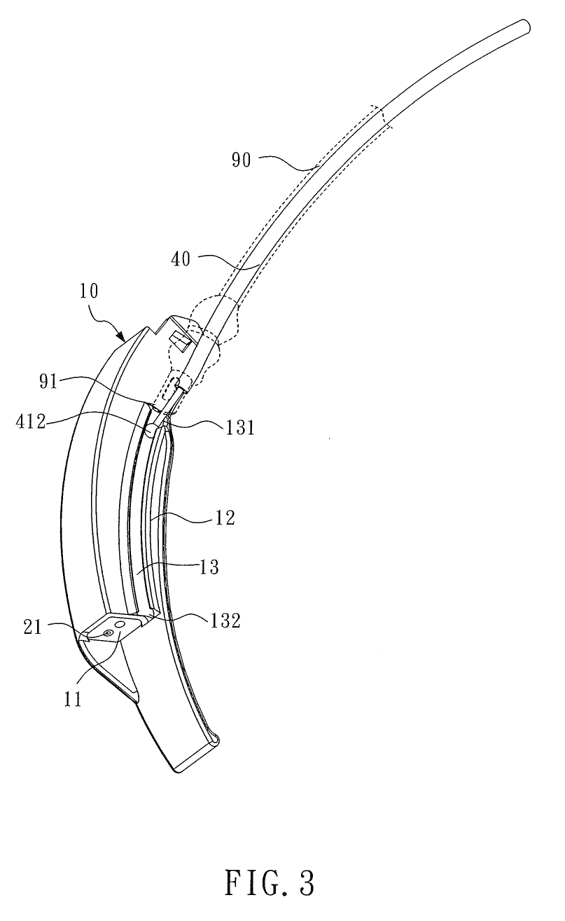

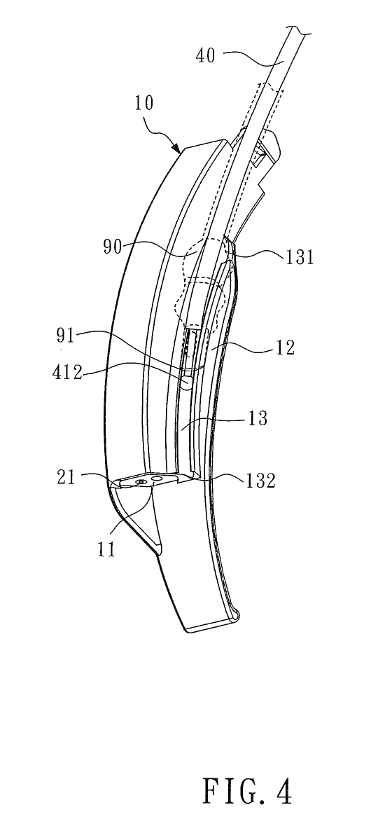

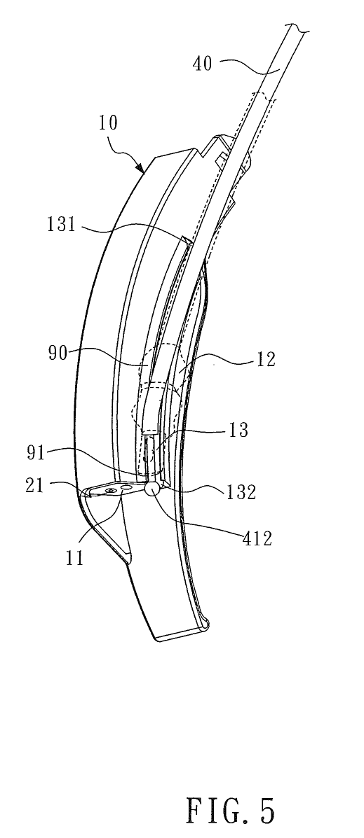

Please refer to FIG. 1, 2A, 2B and FIGS. 3 to 5 respectively for a schematic diagram of one embodiment of the laryngoscope of the present invention; an explosion schematic of the blade, the camera and the guiding stylet; a partial magnification diagram of the guiding stylet; a schematic diagram of the blade, the camera and the guiding stylet; and schematic diagrams of the guiding stylet moving in the guide channel.

As shown in FIGS. 1, 2A and 3, the laryngoscope 1 of the present invention comprises a blade 10, a camera 20, a display 30 and a guiding stylet 40. In the present embodiment, the blade 10 comprises a camera accommodation portion 11, an endotracheal tube passage 12 and a guide channel 13, wherein the guide channel 13 is situated between the camera accommodation portion 11 and the endotracheal tube passage 12. The camera accommodation portion 11 is used for disposing a part of the camera 20. The display 30 is electrically connected to the camera 20 for displaying an image captured by the camera 20. The guiding stylet 40 is situated inside the endotracheal tube 90 for controlling the direction of the endotracheal tube 90 and guiding a tip 91 of the endotracheal tube 90 to keep moving toward the direction of the guide channel 13 when the endotracheal tube 90 is moving in the endotracheal tube passage 12. Thus, when the endotracheal tube 90 separates from the endotracheal tube passage 12, the direction of the tip 91 of the endotracheal tube 90 is maintained due to inertia. Therefore, puncturing of the patient's tissue by the tip of the guiding stylet 40 or the front end of the endotracheal tube 90 can be avoided, and the accuracy of the insertion of the endotracheal tube 90 into the trachea can also be improved.

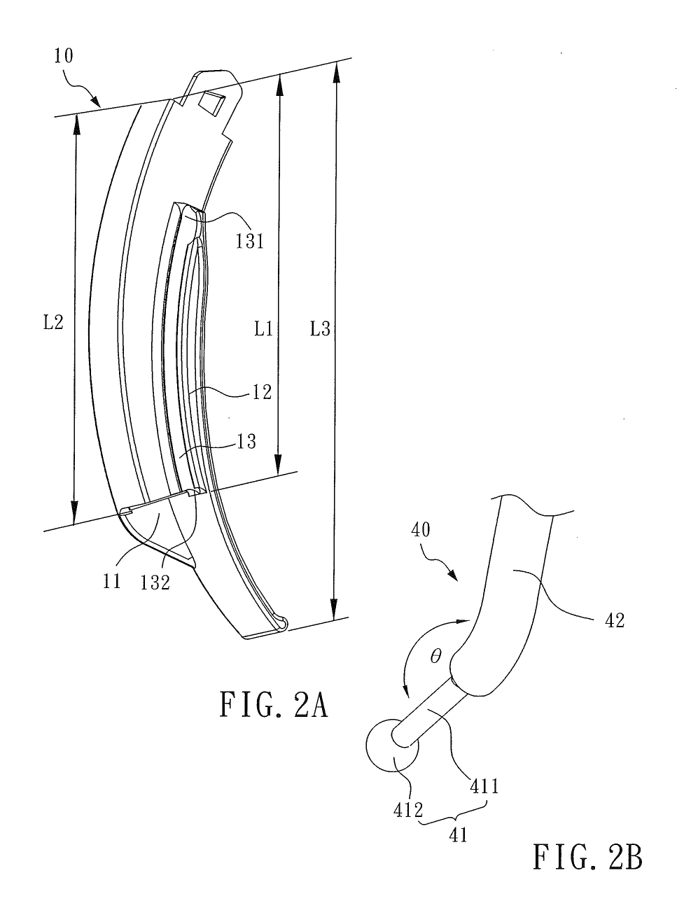

As shown in FIGS. 1, 3 and 4, one part of the camera 20 is situated in the camera accommodation portion 11. The camera 20 comprises a capturing end 21 and an electrical connection end 22. The display 30 is electrically connected to the camera 20. As shown in FIG. 2B, the guiding stylet 40 comprises a guiding unit 41 which is detachably situated in the guide channel 13 and movable relative to the guide channel 13 (as shown in FIGS. 3 to 5). It is noted that the camera 20 and the display 30 are currently known techniques and that the two elements are not modified by the present invention; thus, the details are not described here.

As shown in FIGS. 1, 2A and 2B, in the present embodiment, the blade 10 has a tongue piece 14 which comprises an top surface 141 and a bottom surface 142, wherein the camera accommodation portion 11, the endotracheal tube passage 12 and the guide channel 13 contact the bottom surface 142, and the guide channel 13 is situated between the camera accommodation portion 11 and the endotracheal tube passage 12. As shown in FIG. 2A, the guide channel 13 has a first opening 131 and a second opening 132, wherein a radius of the first opening 131 is larger than a radius of the second opening 132; thus, the guiding unit 41 is able to be situated inside the guide channel 13 and to move within the guide channel 13.

As shown in FIGS. 1 and 2B, in the present embodiment, the guiding stylet 40 further comprises a main body 42, wherein when the guiding stylet 40 is situated inside the endotracheal tube 90, the majority of the main body 42 is situated inside the endotracheal tube 90. The guiding unit 41 comprises a connector 411 and a guiding unit 412, wherein the two opposite ends of the connector 411 are respectively connected to one end of the main body 42 and to the guiding unit 412. As shown in FIG. 2B, in the present embodiment, the guiding unit 412 is spherical shape which is able to be detachably situated in the guide channel 13 and is movable relative to the guide channel 13 (as shown in FIGS. 3 to 5). As shown in FIG. 2B, in the present embodiment, there is an angle θ between an axial direction of the connector 42 and an axial direction of the main body 42, wherein the angle θ is from 175 degrees to 95 degrees, but the present invention is not limited to the aforementioned degrees as long as the connector 411 is not disposed on the same axis as the main body 42 to allow the guiding unit 412 to be situated in the direction close to the guide channel 13.

As shown in FIG. 2A, in the present embodiment, the guide channel 13 has a first length L1, and the camera accommodation portion 11 where the camera 20 is situated has a second length L2, wherein the first length L1 is equal to or longer than the second length L2. This design can ensure that the operator will push the guiding stylet 40 and the endotracheal tube 90 after clearly seeing the patient's trachea so as to improve the accuracy of intubation. This design also prevents failed intubation or accidental injury to the patient due to the endotracheal tube 90 blocking the field of vision of the camera 20 such that the operator cannot see the correct location of the patient's trachea. Furthermore, as shown in FIG. 2A, the tongue piece 14 has a third length L3, wherein the third length L3 is longer than the first length L1.

As shown in FIGS. 3 to 5, the guiding unit 412 of the guiding stylet 40 enters the guide channel 13 via the first opening 131 and moves along the guide channel 13 to separate from the guide channel 13 via the second opening 132. Thus, the tip 91 of the endotracheal tube 90 continues to move along the guide channel 13 to prevent the front end of the endotracheal tube 90 from puncturing the patient, and the accuracy of the insertion of the endotracheal tube 90 into the trachea can also be improved.

By the engagement of the guide channel 13 with the guiding stylet 40 of the laryngoscope 1 of the present invention, the guide unit 41 can be inserted into the first opening 131 of the guide channel 13 and moved to the second opening 132 and separate from the guide channel 13 after the operator clearly sees the correct position of the patient's trachea. Thus, the accuracy of insertion of the endotracheal tube 90 into the trachea can also be improved, and puncturing of the patient's tissue by the tip of the guiding stylet 40 or the front end of the endotracheal tube 90 can be avoided when the endotracheal tube 90 is moving in the endotracheal tube passage 12.

It is noted that the above-mentioned embodiments are only for illustration. It is intended that the present invention cover modifications and variations of this invention provided they fall within the scope of the following claims and their equivalents. Therefore, it will be apparent to those skilled in the art that various modifications and variations can be made to the structure of the present invention without departing from the scope of the invention.

What is claimed is:

1. A laryngoscope for placing an endotracheal tube into a body, the laryngoscope comprising:
a blade comprising a camera accommodation portion, an endotracheal tube passage and a guide channel, wherein the guide channel is situated between the camera accommodation portion and the endotracheal tube passage;
a camera, wherein one part of the camera is situated at the camera accommodation portion;
a display electrically connected to the camera; and
a guiding stylet situated inside the endotracheal tube and comprising a guiding unit, wherein the guiding unit is protruded from the endotracheal tube and is detachably situated inside the guide channel and movable relative to the guide channel.

2. The laryngoscope as claimed in claim 1, wherein the blade comprises a tongue piece having a bottom surface, wherein the camera accommodation portion, the endotracheal tube passage and the guide channel all contact the bottom surface.

3. The laryngoscope as claimed in claim 2, wherein the guide channel has a first length and the camera accommodation portion where the camera is situated has a second length, wherein the first length is equal to or longer than the second length.

4. The laryngoscope as claimed in claim 3, wherein the tongue piece has a third length, wherein the third length is longer than the first length.

5. The laryngoscope as claimed in claim 1, wherein the guide channel has a first opening and a second opening, wherein a radius of the first opening is larger than a radius of the second opening.

6. The laryngoscope as claimed in claim 5, wherein the guiding stylet comprises a main body and the guiding unit comprises a connector and a guiding unit, wherein two opposite ends of the connector are respectively connected to one end of the main body and to the guiding unit.

7. The laryngoscope as claimed in claim 6, wherein the guiding unit is a spherical shape.

8. The laryngoscope as claimed in claim 6, wherein an axial direction of the connector has an angle with respect to an axial direction of the main body.

9. The laryngoscope as claimed in claim 8, wherein the angle is from 175 degrees to 95 degrees.

10. The laryngoscope as claimed in claim 2, wherein the guide channel has a first opening and a second opening, wherein a radius of the first opening is larger than a radius of the second opening.

11. The laryngoscope as claimed in claim 10, wherein the guiding stylet comprises a main body and the guiding unit comprises a connector and a guiding unit, wherein two opposite ends of the connector are respectively connected to one end of the main body and to the guiding unit.

12. The laryngoscope as claimed in claim 11, wherein the guiding unit is a spherical shape.

13. The laryngoscope as claimed in claim 11, wherein an axial direction of the connector has an angle with respect to an axial direction of the main body.

14. The laryngoscope as claimed in claim 13, wherein the angle is from 175 degrees to 95 degrees.

15. The laryngoscope as claimed in claim 3, wherein the guide channel has a first opening and a second opening, wherein a radius of the first opening is larger than a radius of the second opening.

16. The laryngoscope as claimed in claim 15, wherein the guiding stylet comprises a main body and the guiding unit comprises a connector and a guiding unit, wherein two opposite ends of the connector are respectively connected to one end of the main body and to the guiding unit.

17. The laryngoscope as claimed in claim 16, wherein the guiding unit is a spherical shape.

18. The laryngoscope as claimed in claim 16, wherein an axial direction of the connector has an angle with respect to an axial direction of the main body.

19. The laryngoscope as claimed in claim 18, wherein the angle is from 175 degrees to 95 degrees.
